(19) United States
(12) Patent Application Publication
(10) Pub. No.: US 2002/0137763 A1
(43) Pub. Date: Sep. 26, 2002

(54) (5R)-(methylamino)-5,6-dihydro-4H-imidazo[4,5,1-ij]quinoline-2(1H)-thione

(76) Inventors: Brad A. Acker, Kalamazoo, MI (US); Richard Frederick Heier, Kalamazoo, MI (US); Alan Q. Jin, Kalamazoo, MI (US); Malcolm Wilson Moon, Kalamazoo, MI (US)

Correspondence Address: Pharmacia & Upjohn Company, Global Intellectual Property, 301 Henrietta Street, Kalamazoo, MI, 49001, US

(21) Appl. No.: 09/838,054

(22) Filed: Apr. 19, 2001

Related U.S. Application Data
(60) Provisional application No. 60/199,954, filed on Apr. 27, 2000.
(60) Provisional application No. 60/234,101, filed on Sep. 21, 2000.

(51) Int. Cl. A61K 31/4745; C07D 471/04

(52) U.S. Cl. 514/292; 546/84

(57) ABSTRACT

The present invention is a novel pharmaceutical agent, (5R)-5-(methylamino)-5,6-dihydro-4H-imidazo[4,5,1-ij]quinoline-2(1H)-thione

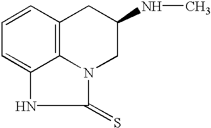

and pharmaceutically acceptable salts thereof.

### CROSS-REFERENCE TO RELATED APPLICATIONS

This application claims the benefit of the following provisional applications: U.S. Serial No. 60/199954, filed Apr. 27, 2000, and U.S. Serial No. 60/234101, filed Sep. 21, 2000, under 35 USC 119(e)(i).

### BACKGROUND OF THE INVENTION

1. Field of the Invention

The present invention is a novel compound which is useful in treating Parkinson's Disease and various sexual dysfunctions.

2. Description of the Related Art

U.S. Pat. No. 5,273,975 generically discloses (5R)-5-(methylamino)-5,6-dihydro-4H-imidazo[4,5,1-ij]quinoline-2(1H)-thione. There is no example or specific mention of this compound.

PCT/US00/00505 discloses (5R)-(methylamino)-5,6-dihydro-4H-imidazo[4,5,1-ij]quinolin-2(1H)-one (EXAMPLE 6) and (5R)-(methylamino)-5,6-dihydro-4H-imidazo[4,5,1 -ij]quinolin-2(1 H)-one maleate (EXAMPLE 7) as well as process to prepare these compounds.

### SUMMARY OF INVENTION

Disclosed is (5R)-(methylamino)-5,6-dihydro-4H-imidazo[4,5, 1 -ij]quinoline-2(1H)-thione, a compound of the formula

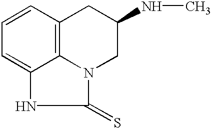

and pharmaceutically acceptable salts thereof.

Also disclosed is a process claim 9

### DETAILED DESCRIPTION OF THE INVENTION

U.S. Pat. No. 5,273,975 generically discloses and claims (5R)-5-(methylamino)-5,6-dihydro-4H-imidazo[4,5,1-ij]quinoline-2(1H)-thione. However, there is no example or identification of this compound.

(5R)-(methylamino)-5,6-dihydro-4H-imidazo[4,5,1-ij]quinoline-2( l H)-thione (VIII) is preferably made from the corresponding non-thio analog, (5R)-(methylamino)-5,6-dihydro-4H-imidao(4,5,1-ij)quinolin-(2H)-one (VII). (5R)-(Methylamino)-5,6-dihydro-4H-imidao(4,5,1-ij)quinolin-(2H)-one (VII) is preferably prepared by the process of

PREPARATION 1 and EXAMPLEs 1-6, see CHART A.

In the process of transforming (5R)-(methylamino)-5,6-dihydro-4H-imidao(4,5,1-ij)quinolin-(2H)-one (VII) into (5R)-(methylamino)-5,6-dihydro4H-imidazo[4,5,1-ij]quinoline-2(1H)-thione (VIII), either the free base or pharmaceutically acceptable salt thereof of the starting material can be used. Pharmaceutically acceptable salts include salts of both inorganic and organic acids. The pharmaceutically acceptable salts are preferred over the corresponding free amines since they produce compounds which are more water soluble and more crystalline. The preferred pharmaceutically acceptable salts include salts of the following acids hydrochloric, hydrobromic, sulfuric, phosphoric, nitric, citric, methanesulfonic CH₃—(CH₂)ₙₗ—COOH where n₁ is 0 thru 4, HOOC—(CH₂)n₁—COOH where n is as defined above, HOOC—CH═CH—COOH, φ—COOH. For other acceptable salts, see Int. J. Pharm., 33, 201-217 (1986).

Regardless of whether the free base or pharmaceutically acceptable salt of (5R)-(methylamino)-5,6-dihydro-4H-imidazo(4,5,1 -ij)quinolin-(2H)-one (VII) is used as the starting material the product is the free base form of (5R)-5-(methylamino)-5,6-dihydro-4H-imidazo[4,5,1-ij]quinoline-2(1H)-thione (VIII). The free base of (5R)-5-(methylamino)-5,6-dihydro-4H-imidazo[4,5,1-ij]quinoline-2(1H)-thione (VIII) is then converted to the corresponding pharmaceutically acceptable salt (IX) as desired.

The preferred method of transforming (5R)-(methylamino)-5,6-dihydro-4H- imidao(4,5,1-ij)quinoline-(2H)-one (VII) into (5R)-5-dihydro-4H- imidazo[4,5,1-ij]quinoline-2(1H)-thione (VIII) is set forth in EXAMPLE 11.

(5R)-5-(methylamino)-5,6-dihydro-4H-imidazo[4,5,1-ij]quinoline-2(1H)-thione (VIII) and the pharmaceutically acceptable salts thereof (IX) are useful as pharmaceutical agents as disclosed in U.S. Pat. No. 5,273,975.

### DEFINITIONS AND CONVENTIONS

The definitions and explanations below are for the terms as used throughout this entire document including both the specification and the claims.

### DEFINITIONS

All temperatures are in degrees Celsius.

TLC refers to thin-layer chromatography.

HPLC refers to high pressure liquid chromatography.

Saline refers to an aqueous saturated sodium chloride solution.

Chromatography (column and flash chromatography) refers to purification/separation of compounds expressed as (support, eluent). It is understood that the appropriate fractions are pooled and concentrated to give the desired compound(s).

IR refers to infrared spectroscopy.

CMR refers to C-13 magnetic resonance spectroscopy, chemical shifts are reported in ppm (δ) downfield from TMS.

NMR refers to nuclear (proton) magnetic resonance spectroscopy, chemical shifts are reported in ppm (δ) downfield from tetramethylsilane.

−φ refers to phenyl (C₆H₅).

[α]_{D}²⁵ refers to the angle of rotation of plane polarized light (specific optical rotation) at 25° with the sodium D line (589A).

MS refers to mass spectrometry expressed as m/e, m/z or mass/charge unit. [M+H]⁺ refers to the positive ion of a parent plus a hydrogen atom. EI refers to electron impact. CI refers to chemical ionization. FAB refers to fast atom bombardment.

Pharmaceutically acceptable refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability.

When solvent pairs are used, the ratios of solvents used are volume/volume (v/v).

When the solubility of a solid in a solvent is used the ratio of the solid to the solvent is weight/volume (wt/v).

### EXAMPLES

Without further elaboration, it is believed that one skilled in the art can, using the preceding description, practice the present invention to its fullest extent. The following detailed examples describe how to prepare the various compounds and/or perform the various processes of the invention and are to be construed as merely illustrative, and not limitations of the preceding disclosure in any way whatsoever. Those skilled in the art will promptly recognize appropriate variations from the procedures both as to reactants and as to reaction conditions and techniques.

### PREPARATION 1

### (R)-Naproxen chloride

R-naproxen (Can. J. Chem., 72(1), 142-5 (1994), 260 g), methylene chloride (3.33 kg) and DMF (8.2 ml) are added to a reactor. Oxalyl chloride (191.8 g) is slowly added to this mixture. After addition of the oxalyl chloride, the slurry is stirred at 5 to 10° and then slowly warmed to 20-25°. The resulting mixture is concentrated to remove the methylene chloride, branched octane is added to the concentrate and the mixture is again concentrated. More branched octane is added to the concentrate and the mixture is cooled to 0° and stirred to crystallize. The crystal slurry is filtered, the crystal cake is washed with octane and dried at 20-25° to obtain the title compound.

The filtrate from the first crop is concentrated, branched octane is added and the mixture is cooled and stirred to obtain a second crop of the title compound. The slurry is filtered, the crystal cake is washed with branched octane and dried at 20-25°.

### EXAMPLE 1

### 1-Benzyl-4H-imidazo[4,5,1-ij]quinolin-2(1H)-one (II)

A mixture of 4H-imidazo[4,5,1-ij]quinolin-2(1H)-one (I, J. Heterocyclic Chem., 19, 837-49 (1982), 1.0 g, 5.8 mmol) in DMF (10 ml) is cooled to 0° and treated with potassium t-butoxide in THF (1.98 M, 3.2 ml, 6.3 mmol) maintaining the reaction temperature at 0°. The resulting mixture is stirred at 0° for 10 minutes. Benzyl bromide (0.73 ml, 6.1 mmol) is then added while maintaining the reaction temperature at methyl t-butyl ether (MTBE) from water followed by several water washes. The MTBE phase is concentrated under reduced pressure. The concentrate is cooled to 0°, filtered and washed two times with 0° MTBE. The product is dried at 50° under reduced pressure with a nitrogen purge to give the title compound, CMR (CDCl₃, 100 MHz) 153.78, 136.44, 128.69, 127.67, 127.60, 126.73, 125.86, 122.90, 122.78, 121.28, 116.92, 116.17, 108.36, 44.95 and 42.37 φ.

### EXAMPLE 2

### (5R,6R)-1-Benzyl-5-bromo-6-hydroxy-5,6-dihydro-4H-imidazo[4,5,1 - ij]quinolin-2(1H)-one (III)

1-Benzyl-4H-imidazo[4,5,1-ij]quinolin-2(1H)-one (II, EXAMPLE 1, 240 g), acetonitrile (1.086 kg), water (227 ml) and fluoboric acid (48.5%, 13.4 g) are mixed and cooled to 0 to 5°. Dibromantin (163.5 g) is slurried into acetonitrile and is added to the reaction mixture. The reaction is carried out for about 3 hr at 0 to 5°. After the reaction is complete, methyl t-butyl ether is added over about 45 minutes keeping the reaction temperature in the pot below 10°. The slurry is cooled to −10 to −15°, stirred for an hour and then filtered. The product is washed with precooled methyl t-butyl ether, dried with 40° nitrogen to give the title compound, CMR (CDCl₃) 156.0, 137.8, 130.5, 129.6, 129.3, 129.1, 126.6, 123.6, 122.5, 119.6, 110.4,69.9,49.6, 47.7,46.9 and 43.8 δ.

### EXAMPLE 3

### (5S ,6S)- 1-Benzyl-5-bromo-2-oxo- 1,2,5,6-tetrahydro-4H-imidazo[4,5,1-ij]quinolin-6-yl (2R)-(6-methoxy-2-naphthyl)propanoate (IVA) and (5R,6R)-1-benzyl-5-bromo-2-oxo- 1,2,5,6-tetrahydro-4H-imidazo[4,5,1 -ij]quinolin-6-yl (2R)-(6-methoxy-2-naphthyl)propanoate (IVB)

(5R,6R)-1-Benzyl-5-bromo-6-hydroxy-5,6-dihydro-4H-imidazo[4,5,1 -ij]quinolin-2(1H)-one (III, EXAMPLE 2, 143 g), methylene chloride (3,136 g), N-methyl morpholine (100.2 g) and 4-dimethylaminopyridine (497 mg) are added to the reactor and the mixture is cooled to 0 to 5°. (R)-Naproxen chloride (PREPARATION 1, 118.5 g) dissolved in methylene chloride (694 ml) is added to the reactor over about 1 hr and the mixture is stirred at 0 to 50 to complete the reaction. If necessary, additional naproxen chloride is added to complete the reaction. Potassium carbonate solution diluted with water is added to the mixture. The aqueous phase is extracted with methylene chloride and the combined methylene chloride phase is washed with water. The washed mixture is concentrated by vacuum distillation and solvent exchange with ethyl acetate is performed. The concentrate is cooled to −10° and stirred. The crystal slurry is filtered and the crystal cake is washed with precooled methyl t-butyl ether and dried at 50° to give the title compound in solid form, (5S,6S)-1-benzyl-5-bromo-2-oxo- 1,2,5,6-tetrahydro-4H-imidazo[4,5,1-ij]quinolin-6-yl (2R)-2-(6-methoxy-2-naphthyl)propanoate (IVA), CMR (CDCl₃) δ 173.2, 157.8, 153.4, 136.1, 134.6, 133.7, 129.2, 128.8, 127.8, 127.8, 127.6, 127.2, 125.9, 125.9, 125.6, 121.5, 121.4, 119.1, 113.2, 109.0, 105, 105.6, 69.2, 55.3, 45.4, 45.2, 42.5, 41.7 and 18.3.

The undesired isomer, (5R,6R)-1-benzyl-5-bromo-2-oxo- 1,2,5,6-tetrahydro-4H-imidazo[4,5,1-ij]quinolin-6-yl (2R)-2-(6-methoxy-2-naphthyl)propanoate (IVB) is in the filtrate and can be recovered by means well known to those skilled in the art, (5R,6R)- 1-benzyl-5-hydroxy-6-(methylamino)-5,6-dihydro-4H-imidazo[4,5,1 -ij]quinolin-2(1H)-one, CMR (CDCl₃) δ 173.2, 157.9, 153.4, 136.1, 135.0, 133.8, 129.2, 128.9, 128.8, 127.8, 127.6, 127.4, 125.8, 125.8, 125.7, 121.6, 121.5, 119.3, 113.1, 109.1, 105.7,68.7, 55.3, 45.3, 45.2, 42.2, 41.3 and 18.1.

### EXAMPLE 4

### (5R,6R)-1-Benzyl-5-hydroxy-6-(methylamino)-5,6-dihydro-4H-imidazo[4,5,1-ij]quinolin-2(1H)-one (V)

(5S,6S)-1-Benzyl-5-bromo-2-oxo- 1,2,5,6-tetrahydro4H-imidazo[4,5,1-ij]quinolin-6-yl (2R)-2-(6-methoxy-2-naphthyl)propanoate (IVA, EXAMPLE 3, 110 g) is slurried in acetonitrile (1,297 g). After adding aqueous methylamine (40 wt %, 327 g) the reaction is carried out for about 12 hr at about 30°. After the reaction is complete, the mixture is concentrated and ethyl acetate is added. Dilute hydrochloric acid is added to make the water-soluble salt of the title compound. The byproduct (R-naproxen methylamide impurity) is insoluble in water and stays in the ethyl acetate phase. Further extractions and washes are carried out for better separation of the (naproxen acetamide) impurity with minimum loss of the desired product. Then a sodium hydroxide solution is added to the aqueous phase and the hydrochloride salt of the title compound is converted to the free base. The free base is less soluble in water and is extracted into ethyl acetate. The product mixture is concentrated and solvent exchanged with ethyl acetate to remove water. Crystallization is performed by adding branched chain octane and cooling the mixture. The resulting slurry is filtered, washed and dried at 50° to give the title compound, CMR (CDCl₃) δ 153.7, 136.3, 128.7, 127.8, 127.7, 125.7, 121.3, 119.9, 118.6, 107.5, 66.2, 60.1, 45.1, 42.6 and 34.0.

### EXAMPLE 5

### (7aS,8aR)4-Benzyl-8-methyl-7,7a,8,8a-tetrahydroazireno[2,3-c]imidazo[4,5,1-ij]quinolin-5(4H)-one (VI)

(5R,6R)-1-Benzyl-5-hydroxy-6-(methylamino)-5,6-dihydro4H-imidazo[4,5,1-ij]quinolin-2(1H)-one (V, EXAMPLE 4,70 g) and THF (1,389 g) is concentrated to remove any by distillation as a precaution due to reactivity of n-butyllithium towards water. The mixture is cooled to about −10° and n-butyyllithium is added to make the lithium salt of the starting material with formation of n-butane byproduct in an exothermic reaction. Benzenesulfonyl chloride is added slowly to make benzenesulfonate in an exothermic reaction. The reaction mixture is warmed to 20-25° to complete the reaction. Aqueous potassium carbonate solution is added to scavenge the benzenesulfonic acid and the mixture is stirred to allow crystallization. Water is added to complete crystallization, the slurry is stirred, cooled and filtered. The crystal cake is washed with water followed by branched chain octane and dried at 40 to 50° to give the title compound, CMR (CDCl₃) δ 154.1, 136.3, 128.6, 127.9, 127.6, 124.3, 120.7, 119.7, 107.4, 46.7, 44.9, 40.7, 38.1 and 37.6.

### EXAMPLE 6

### (5R)-(Methylamino)-5,6-dihydro-4H-imidazo[4,5,1-ij]quinolin-2(1H)-one (VII)

A mixture of (7aS,8aR)-4-benzyl-8-methyl-7,7a,8,8a-tetrahydroazireno[2,3-c]imidazo[4,5,1-ij]quinolin-5(4H)-one (VI, EXAMPLE 5, 40 g) t-amyl alcohol (42.4 g) and anhydrous ammonia (1,200 g) is treated with lithium at −33° . After the lithium addition is complete, the reaction mixture changes from a yellow slurry to a dark blue mixture. This dark blue mixture is stirred for 30-60 minutes and then quenched with the addition of water. The cooling is removed from the condenser and the ammonia is allowed to evaporate. The residue is dissolved in methanol. This mixture is then concentrated to dryness to give the title compound, which is carried on directly to the next step without isolation.

### EXAMPLE 7

### (5R)-(Methylamino)-5,6-dihydro-4H-imidazo[4,5,1-ij]quinoline-2(1H)-thione (VIII)

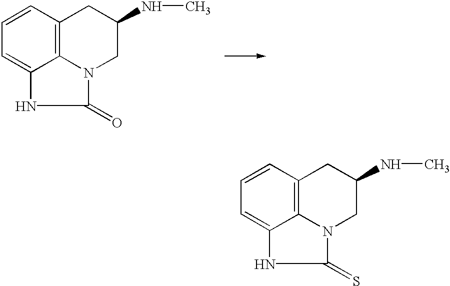

A mixture of (5R)-(methylamino)-5,6-dihydro-4H-imidazo[4,5,1-ij]quinolin-2(1H)-one (VIII, EXAMPLE 6, 15.0 g, 73.8 mmol) and tetraphosphorus decasulfide (36.1 g, 81.2 mmol) in pyridine (300 mL) is heated in a 125° oil bath under nitrogen. The reaction is stirred for 5 hr. The mixture is cooled to 20-25° and the pyridine is removed under reduced pressure. Sodium hydroxide (2.2 N, 200 mL) is added and a vigorous reaction ensues. Additional sodium hydroxide (1 N) is added until a solution is formed. The solution is saturated with sodium chloride and extracted with methylene chloride (2.5 L, in portions). The organic phase is absorbed onto silicon dioxide (40 g) and purified via column chromatography (silicon dioxide, 225 g; methanol/methylene chloride, 3.5-5.0/96.5-95). The appropriate fractions are pooled and concentrated. The material is recrystallized from methanol/ethyl acetate/hexanes to give the title compound, mp=210-213°; IR (drift) 2940, 2907, 2884, 1483, 1458, 1391, 1366, 1354, 1254, 1239, 1229, 895, 762, 734 and 630 cm⁻¹; NMR (300 MHz, CDCl₃) δ 7.12, 7.03, 7.00, 4.30, 3.96, 3.30-3.50, 3.15, 2.88 and 2.57; MS (EI) m/z 219 (M⁺), 190, 189, 187, 186, 164, 163, 155, 145; HRMS (FAB) calculated for C₁₁H₁₃N₃S (MH⁺)=220.0908, found=220.0904.

### EXAMPLE 8

### (5R)-(Methylamino)-5,6-dihydro-4H-imidazo[4,5,1-ij]quinoline-2(1H)-thione maleate (IX)

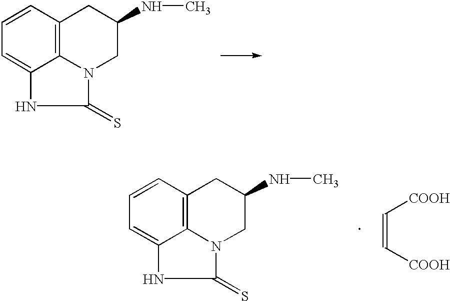

A solution of maleic acid (0.317 g, 2.36 mmol) in a minimal amount of methanol (˜1 mL) is added to a mixture of (5R)-(methylamino)-5,6-dihydro4H-imidazo[4,5,1-ij]quinoline-2(1H)-thione (VIII, EXAMPLE 7, 0.493 g, 2.25 mmol) in methylene chloride. The resulting solid is collected by filtration to give the title compound; mp=195-196°; [α]²⁵D=−60° (c 0.93, methanol); IR (drift) 3140, 3112. 3060, 2969, 1627, 1619, 1568, 1481, 1455, 1398, 1389, 1361, 1220, 868 and 747 cm⁻¹; NMR (300 MHz, CD₃OD) δ 7.20-7.30, 7.10-7.20, 6.26, 4.49, 4.31, 4.05-4.20, 3.28 and 2.83; CMR (100 MHz, DMSO−d₆+CD₃OD) δ 170.4, 169.4, 136.6, 131.1, 130.9, 125.1, 122.1, 116.2, 109.6, 53.9, 43.1, 31.9 and 27.2; MS (ESI) m/z=220.1 (MH⁺).

### EXAMPLE 9

### (5R)-(methylamino)-5,6-dihydro-4H-imidazo[4,5,1-ij]quinolin-2(1H)-one maleate (VII)

(5R)-(methylamino)-5,6-dihydro-4H-imidazo[4,5,1-ij]quinolin-2( 1H)-one (VII, EXAMPLE 6, 28.0 g) is dissolved in water and the pH is adjusted to 10 with the addition of hydrochloric acid. The mixture is applied in portions to an XAD-16 resin column which is eluted first with water and then with ethanol. The inorganic salts are eluted from the column first with the desired product eluted with the ethanol. The ethanol eluate from the column is treated with maleic acid and the water level is lowered through azeotropic distillation of the ethanol. The precipitated product is isolated by filtration, rinsed with ethyl acetate and dried to give the title compound, CMR (DMSO-d₆) δ 167.6, 153.9, 136.4, 127.1, 121.5, 119.6, 114.1, 107.5, 51.9, 31.3 and 26.5.

### EXAMPLE 10

### (5R)-(Methylamino)-5,6-dihydro-4H-imidazo[4,5,1 -ij]quinoline-2(1H)-one hydrochloride (VIII)

Concentrated hydrochloric acid (425 ml) is added to a slurry of (5R)-(methylamino)-5,6-dihydro-4H-imidazo[4,5,1 -ij]quinolin-2(1H)-one maleate (VII, EXAMPLE 9, 850 g) in ethanol (7.65 liters). The mixture is stirred at 20-25° and concentrated while adding additional ethanol. The product is isolated by filtration and the cake is washed with ethanol and dried to give the title compound, [α]²⁵D=−35° (water); UV 206 (59400), 227 (7020), 279 (5540), 282 (5570); NMR (400 MHz, D₂O) δ 7.05-7.09, 6.95-6.99, 4.73, 4.09-4.13, 3.96-4.01, 3.88-3.93, 2.94-3.25 and 2.76; CMR (100 MHz, D₂O) δ 155.25, 126.26, 126.08, 123.08, 120.88, 114.27, 108.97, 52.60, 39.72, 31.49 and 26.34.

### EXAMPLE 11

### (5R)-5-(Methylamino)-5,6-dihydro-4H-imidazo[4,5,1-ij]quinoline-2(1H)-thione maleate (IX)

A solution of (5R)-5-(Methylamino)-5,6-dihydro-4H-imidazo[4,5,1-ij]quinoline-2(1H)-one hydrochloride (VIII, EXAMPLE 10, 11.0 kg) and phosphorous pentasulfide (20.4 kg) in pyridine is refluxed until the reaction is complete. The reaction is quenched with aqueous potassium hydroxide. The solution is vacuum distilled, and diluted with water. Concentrated hydrochloric acid is added to lower the pH to 10.0-10.5, and the solution is extracted with a mixture of n-butyl alcohol/ethyl acetate (20/80) at about 70°. The organic extracts are vacuum distilled while adding methanol. The slurry is mixed with a solution of maleic acid (6.0 kg) in methanol. The solution is clarified by filtration, and the filtrate is vacuum concentrated while adding ethanol. The resulting crystalline product is isolated by filtration, and the cake is washed with ethanol, and dried to give the title compound, [α]²⁵D=−56° (water); UV 215 (26800), 248 (18000), 299 (21800), 307 (29800); NMR (400 MHz, D₂O) δ 7.33-7.37, 7.22-7.26, 6.34, 4.52-4.56, 4.35-4.40, 4.26-4.30. 3.50-3.55, 3.36-3.40 and 25 2.95; CMR (100 MHz, D₂O) δ 171.02, 165.33, 134.80, 129.30, 124.93, 122.02, 115.58, 109.65, 52.92, 42.39, 31.48 and 26.22.

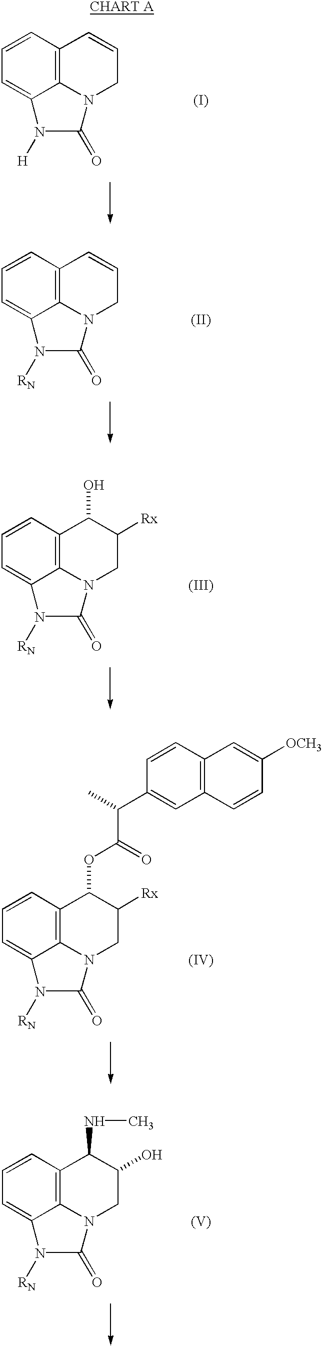

1. A compound of the formula 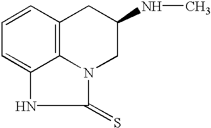
and pharmaceutically acceptable salts thereof.

2. A compound according to claim 1 where the pharmaceutically acceptable salts are selected from the group consisting of salts of the following acids hydrochloric, hydrobromic, sulfuric, phosphoric, nitric, citric, methanesulfonic, CH₃—(CH₂)ₙ₁—COOH where n₁ is 0 thru 4, HOOC—(CH₂)n₁—COOH where n is as defined above, HOOC—CH═CH—COOH and φ—COOH.

3. A compound according to claim 1 which is

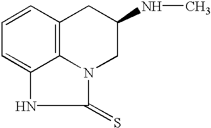

4. A compound according to claim 3 which is

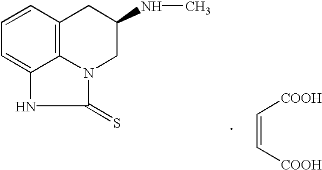

5. (5R)-5-(methylamino)-5,6-dihydro-4H-imidazo[4,5,1-ij]quinoline-2(1H)-thione and pharmaceutically acceptable salts thereof.

6. A compound according to claim 5 where the pharmaceutically acceptable salts are selected from the group consisting of consisting of salts of the following acids hydrochloric, hydrobromic, sulfuric, phosphoric, nitric, citric, methanesulfonic, CH₃—(CH₂)ₙ₁—COOH where n₁ is 0 thru 4, HOOC—(CH₂)ₙ₁—COOH where n is as defined above, HOOC—CH═CH—COOH and φ—COOH.

7. A compound according to claim 5 which is (5R)-5-(methylamino)-5,6-dihydro-4H-imidazo[4,5,1-ij]quinoline-2( 1H)-thione.

8. A compound according to claim 7 which is (5R)-5-(methylamino)-5,6-dihydro-4H-imidazo[4,5,1-ij]quinoline-2( 1H)-thione maleate.

9. A process for the preparation of (5R)-(methylamino)-5,6-dihydro-4H-imidazo[4,5,1-ij]quinoline-2(1H)-thione which comprises:
(1) contacting (5R)-(methylamino)-5,6-dihydro4H-imidazo[4,5,1-ij]quinoline-2(1H)-one or pharmaceutically acceptable salts thereof with tetraphosphorous decasulfide and
(2) heating to more than 100°.

10. A process according to claim 9 where the heating is to about 125°.

11. A process according to claim 9 where the solvent is pyridine.

12. A process according to claim 9 where the (5R)-(methylamino)-5,6-dihydro4H-imidazo[4,5,1-ij]quinoline-2(1H)-one is present as the free base.

13. A process according to claim 9 where the pharmaceutically acceptable salt is selected from the group consisting of the salts of the following acids hydrochloric, hydrobromic, sulfuric, phosphoric, nitric, citric, methanesulfonic CH₃—(CH₂)ₙ₁—COOH where n₁ is 0 thru 4, HOOC—(CH₂)n₁—COOH where n is as defined above, HOOC—CH═CH—COOH, φ—COOH.

14. A process according to claim 9 where the (5R)-(methylamino)-5,6-dihydro4H-imidazo[4,5,1-ij]quinoline-2(1H)-one is present as the hydrochloride salt.
